(19) United States
(12) Patent Application Publication
(10) Pub. No.: US 2017/0298981 A1
(43) Pub. Date: Oct. 19, 2017

(54) HINGE ASSEMBLY FOR AN ORTHOPEDIC DEVICE

(71) Applicant: Ossur Iceland ehf, Reykjavik (IS)

(72) Inventor: Sigurdur ÁSGEIRSSON, Foothill Ranch, CA (US)

(21) Appl. No.: 15/485,422

(22) Filed: Apr. 12, 2017

Related U.S. Application Data
(60) Provisional application No. 62/322,362, filed on Apr. 14, 2016.

(51) Int. Cl. F16C 11/04; A61F 5/02; F16C 11/10; A61F 5/01

(52) U.S. Cl. CPC F16C 11/04; F16C 11/10; A61F 5/0123; A61F 5/022; A61F 5/028

(57) ABSTRACT

A hinge assembly for use in an orthopedic or prosthetic system includes first and second connection members arranged to rotate relative to one another about at least one pivot point. At least one shaft member is rotatably attached to the first connection member. A translating member is attached to the at least one shaft member and is operatively connected to or meshes with the second connection member. Rotation of the at least one shaft member drives translation of the translating member along a length of the at least one shaft member. Translation of the translating member along the shaft member drives rotation of the second connection member relative to the first connection member.

### BACKGROUND

Many conventional orthopedic and prosthetic devices require at least one locking hinge for controlling, supporting, immobilizing, or treating muscles, joints, or skeletal parts, which may be weak, ineffective, or injured. For instance, to assist in restoring a human joint to normal, effective function, it may be prescribed by a clinician that the joint be restricted for a period by an orthopedic device including at least one hinge which imposes a fixed pivoted position on the joint. Or an orthopedic device may be needed that permits adjustable angular displacement of the joint, which is retained for a period of time by the orthopedic device and gradually increased to improve the pivotal range of use.

While several locking hinge products exist, they suffer from a number of drawbacks. For instance, many known locking hinges are complicated in design, bulky, and do not provide sufficient motion control. They are also known to fail or undesirably move from a locked pivoted position set by a clinician, increasing the likelihood of injury to a patient. Furthermore, many existing locking hinges are not capable of providing continuous angular adjustment, and are difficult to adjust while under a load.

It can be seen from the foregoing there are many needs for improving on the drawbacks of conventional locking hinges. The embodiments of the present disclosure address many of these aforementioned shortcomings.

### SUMMARY

The disclosure describes various embodiments of a hinge assembly providing a construction and design that facilitates greater control of movement and stronger support for an orthopedic or prosthetic device.

The embodiments described can include a hinge assembly including first and second connection members arranged to rotate relative to one another about at least one pivot point. The hinge assembly is lockable so as to retain the connection members at a selected angle relative to one another and also adjustable so as to modify the angle at which one of the connection members is positioned relative to the other.

A shaft member is rotatably attached to the first connection member and a translating member is threadedly attached to the shaft member. The translating member is substantially restrained from rotation so that rotation of the shaft member results in translation of the translating member along a length of the shaft member. The translating member also is operatively connected to or meshes with the second connection member.

To adjust the angular position between the first and second connection members, the shaft member is rotated relative to the first connection member. This rotation causes the translating member to translate along the shaft member, which, in turn, causes the second connection member to pivot relative to the first connection member. Rotation of the shaft member thus both controls and drives rotation of the second connection via translation of the translating member.

Because of shear friction and/or a high mechanical advantage of the hinge assembly, the hinge assembly can be self-locking. In other words, an input force or torque applied to the second connection member will not move the second connection member, the translating member, or the shaft member. Thus, whatever angle is set between the connection members by the shaft member remains substantially fixed until the shaft member is readjusted. In addition, the self-locking configuration of the hinge assembly allows the hinge assembly to be locked and/or unlocked under a load, increasing safety and ease of use.

Moreover, because rotation of the shaft member and corresponding translation of the translating member drives and controls rotation of the second connection member, the hinge assembly is continuously adjustable within a range of motion defined by the hinge assembly. Furthermore, because the hinge assembly is self-locking, the hinge assembly can have an infinite number of locking positions within the range of motion.

In an embodiment, the hinge assembly defines large and close-fitting contact areas between the translating member and the shaft member, and/or between the translating member and the second connection member. This beneficially helps the hinge assembly to distribute and support greater loads than existing hinges. It also allows the hinge assembly to be made smaller and/or simpler than in the prior art. This can result in a hinge assembly that is less bulky, lighter-weight, and more natural to wear when incorporated in an orthopedic device. The large and close-fitting engagement between the translating member and the shaft member and/or between the translating member and the second connection member also reduces lost motion or play in the hinge assembly, improving both control and strength of the hinge assembly.

### BRIEF DESCRIPTION OF THE DRAWINGS

These and other features, aspects, and advantages of the present disclosure will become better understood regarding the following description, appended claims, and accompanying drawings.

FIG. 1 is a perspective view of an orthopedic device including hinge assemblies according to an embodiment.

FIG. 2 is a perspective view of an orthopedic device including a hinge assembly according to another embodiment.

FIG. 3 is a perspective view of an orthopedic device including hinge assemblies according to another embodiment.

FIG. 4 is a perspective of a hinge assembly according to an embodiment.

FIG. 4A is a cross section view of the hinge assembly in FIG. 4.

FIG. 5 is a perspective view of the hinge assembly in FIG. 4 in a first position.

FIG. 6 is a perspective view of the hinge assembly in FIG. 4 in a second position.

FIG. 7 is a perspective view of the hinge assembly in FIG. 4 in a third position.

FIG. 8 is a partial exploded view of the hinge assembly in FIG. 4.

FIG. 9 is a perspective view of the second connection member in FIG. 4.

FIG. 10 is a perspective view of a hinge assembly according to another embodiment.

FIG. 11 is a partial exploded view of the hinge assembly in FIG. 10.

### DETAILED DESCRIPTION OF VARIOUS EMBODIMENTS

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that unless a term is expressly defined in this application to possess a described meaning, there is no intent to limit the meaning of such term, either expressly or indirectly, beyond its plain or ordinary meaning. Any element in a claim that does not explicitly state “means for” performing a specified function, or “step for” performing a specific function is not to be interpreted as a “means” or “step” clause as specified in 35 U.S.C. §112, paragraph 6.

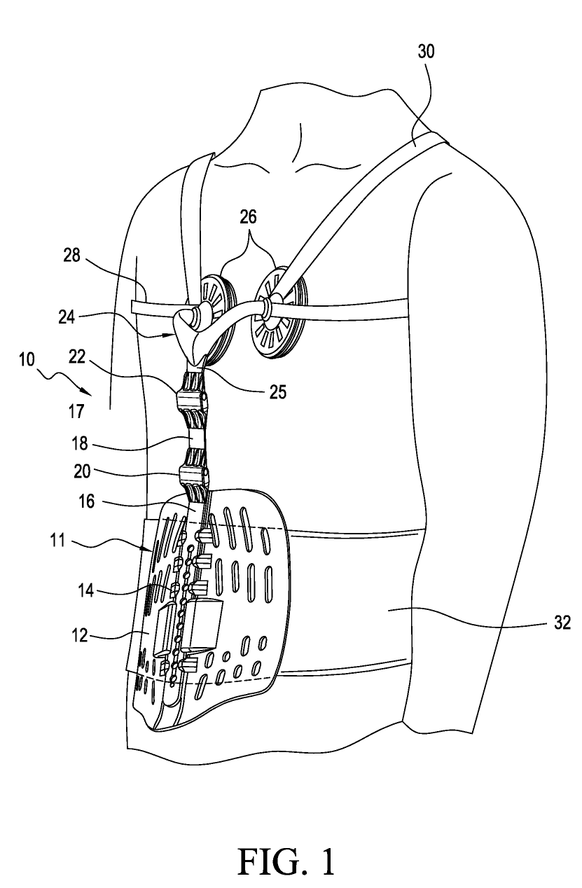

Embodiments of the present disclosure provide a hinge assembly having a construction and design that facilitates greater control of movement and stronger support for an orthopedic or prosthetic device. Moreover, these hinge assembly embodiments can be used in various articles, including, but not limited to, configurations of spinal orthoses, hip orthoses, knee braces, ankle and foot orthoses, ankle braces, wrist braces, cervical collars, elbow braces, prosthetic knees, prosthetic feet, prosthetic ankles, prosthetic joints, or any other suitable article. For example, embodiments of the hinge assembly can be implemented with a spinal orthosis, as shown in FIG. 1. An exemplary spinal orthosis 10 includes an anterior assembly 11 having an anterior plate 12 connected to a torso orthosis 32, a vertical strut 16 selectively secured to the anterior plate 12, a sternal assembly 17 connected to the vertical strut 16 and having a pair of hinge assemblies 20, 22 according to the present disclosure. A pectoral assembly 24 is connected to the sternal assembly and has two pectoral pads 26. Straps 28, 30 are used to secure the pectoral assembly to the chest of the wearer and couple to a posterior assembly belonging to the thoracic assembly.

The sternal assembly 17 is depicted having a dual pivot system. Particularly, the dual pivot system is defined by the hinge assemblies 20, 22, spaced apart by a connecting bar 18. The hinges 20, 22 are pivotable and lockable to a particular orientation.

As seen, the sternal assembly 17 connects to the vertical strut 16 via the lower hinge assembly 20, and the pectoral assembly 24 connects to the sternal assembly 17 via the upper hinge 22 and a stem 25 extending from the pectoral assembly 24. As noted above, the hinge assemblies 20, 22 are fully adjustable so as to modify the angle at which the connecting bar 18 is positioned so as to alleviate pressure on the sternum. The hinge assemblies 20, 22 are also adjustable to adjust the shape and size of the orthosis 10 to the wearer. For instance, the lower hinge assembly 20 can be arranged to move away from the chest and the upper hinge assembly 22 can be arranged to draw the pectoral plates tightly against the chest of the wearer.

With the hinge assemblies 20, 22 adjusted and selectively locked in a fixed pivoted position, any additional adjustments to the orthosis can be conducted to assure a proper, secure fit. An example of the spinal orthosis 10 is described in greater detail in U.S. Pat. No. 9,220,625, granted on Dec. 29, 2015, and commercially available as the MIAMI LUMBAR TLSO by Ossur hf. This disclosure is incorporated by reference and belongs to the assignees of this disclosure.

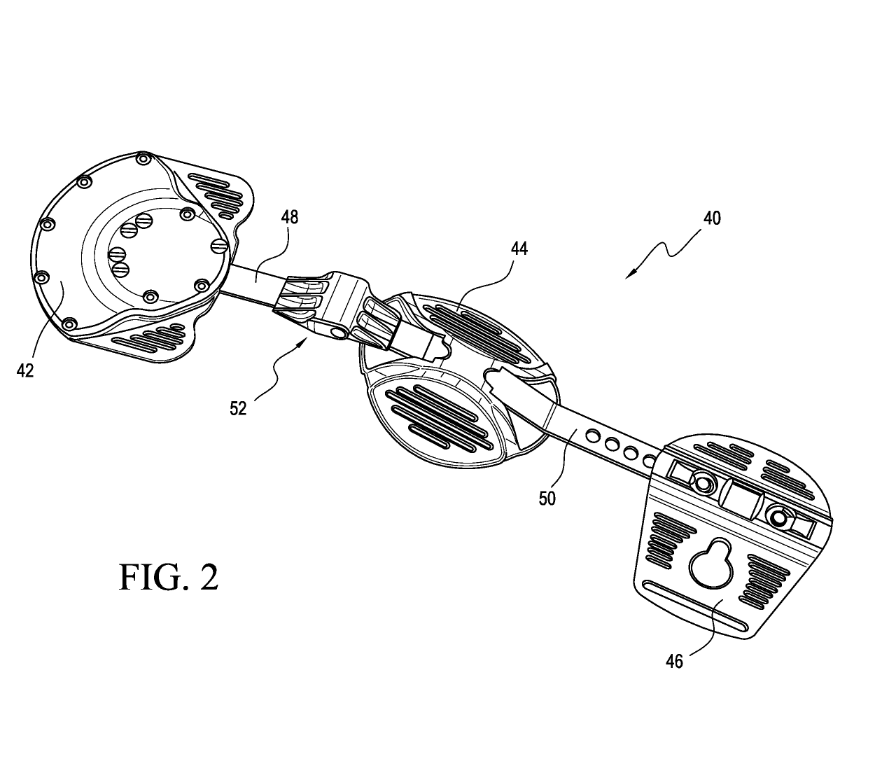

Embodiments of the hinge assembly can also be implemented with a hip orthosis, as shown in FIG. 2. An exemplary hip orthosis 40 can include a pelvic support 42, a trochanter support 44 and a lower support 46. The pelvic support 42 is arranged for placement at or near the pelvis of the wearer, whereas the trochanter support 44 is arranged for placement at or near the trochanter of the femur opposite the femoral head. The lower support 46 can be arranged near and above the knee of the wearer. The pelvic, trochanter and lower supports 42, 44, 46 are connected to one another by a strut assembly comprising at least upper and lower struts 48, 50.

The strut assembly preferably is arranged to act or function as a leaf spring. The strut assembly can be made from metal, plastic, or any other suitable material. Due to the resiliency of the strut assembly, the hip orthosis can exert a force and/or moment on the upper leg, which makes the upper leg abduct, viewed from the front side of the person, preferably independently of the position of the upper leg regarding the waist or trunk.

A lower end of the upper strut 48 is pivotally connected to an upper end of the lower strut 50 via a hinge assembly 52 according to an embodiment of the present disclosure. As previously described, the hinge assembly 52 is self-locking and continuously adjustable within a range of motion defined by the hinge assembly 52. The hinge assembly 52 is arranged to control and/or adjust the angular position of the lower strut 50 relative to the outer surface of the upper strut 48, allowing the hip orthosis to apply adduction or abduction forces to the hip when required. The term “adduction” is defined as being a movement towards the trunk. The term “abduction” is defined as being a movement by which a body part is moved away from the axis of the body. For instance, the hinge assembly 52 can be adjusted to help force adduction or abduction of a patient's hip between about 0 degrees and about 30 degrees, or about 10 degrees and about 20 degrees. Because the hinge assembly 52 can distribute and support greater loads with a smaller and/or simpler construction, the hip orthosis 40 can also be made less bulky, lighter-weight, and more natural to wear. An example of the hip orthosis 40 is described in greater detail in U.S. publication 2014/0207040, published on Jul. 24, 2104, and commercially available as the REBOUND HIP by Ossur hf. This disclosure is incorporated by reference and belongs to the assignees of this disclosure.

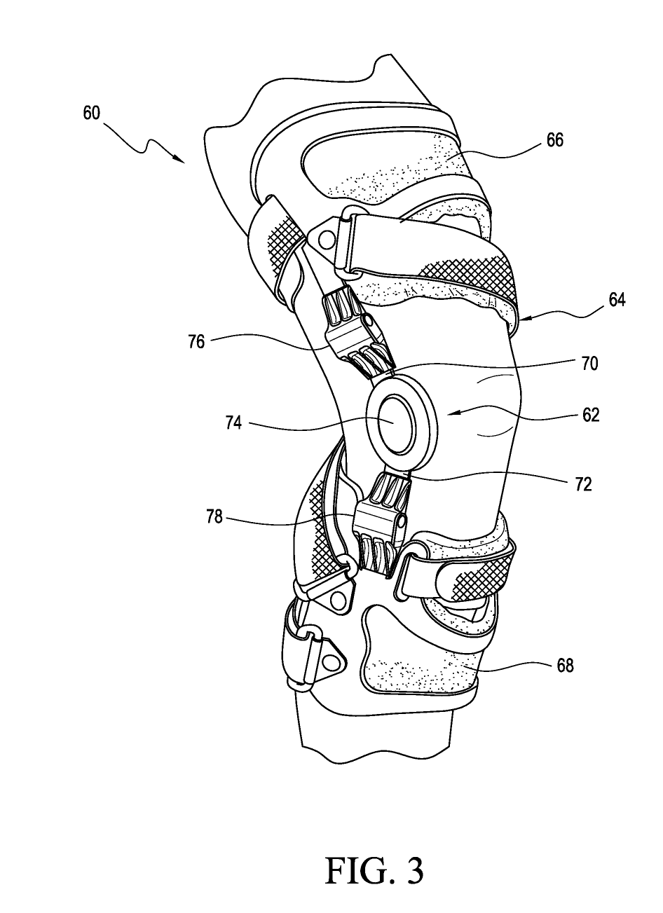

By way of another example, embodiments of the hinge assembly can be implemented with a knee brace as seen in FIG. 3. An exemplary knee brace 60 can include a rigid support structure 62 and a strapping system 64. At the top and bottom ends of the support structure 62 are femoral and tibial cuffs 66, 68, respectively. The support structure 62 includes upper and lower support portions 70, 72 pivotally connected to one another by a polycentric hinge 74 arranged to allow the knee brace 60 to move between flexion and extension. In the illustrated embodiment, each of the upper and lower support portions 70, 72 includes a hinge assembly 76, 78 of the present disclosure. The hinge assemblies 76, 78 are adjustable and lockable to adjust the shape and size of the knee brace 60 to the wearer. The hinge assemblies 76, 78 can also be adjusted and set to apply a corrective force to the leg and/or knee of the wearer. Further, it will be appreciated that at least the hinge assembly 78 can also provide varum or valgum control, helping to offload an affected compartment of the knee.

Another example of a knee brace that can be implemented with embodiments of the hinge assembly is shown and described in U.S. Pat. No. 8,292,838, granted on Oct. 23, 2012, which is incorporated herein by this reference.

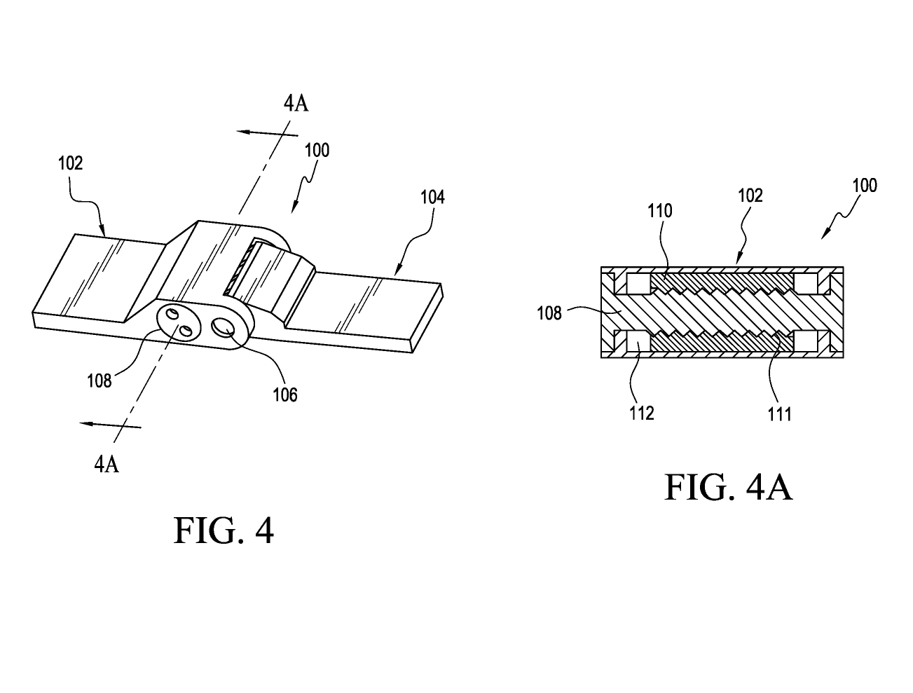

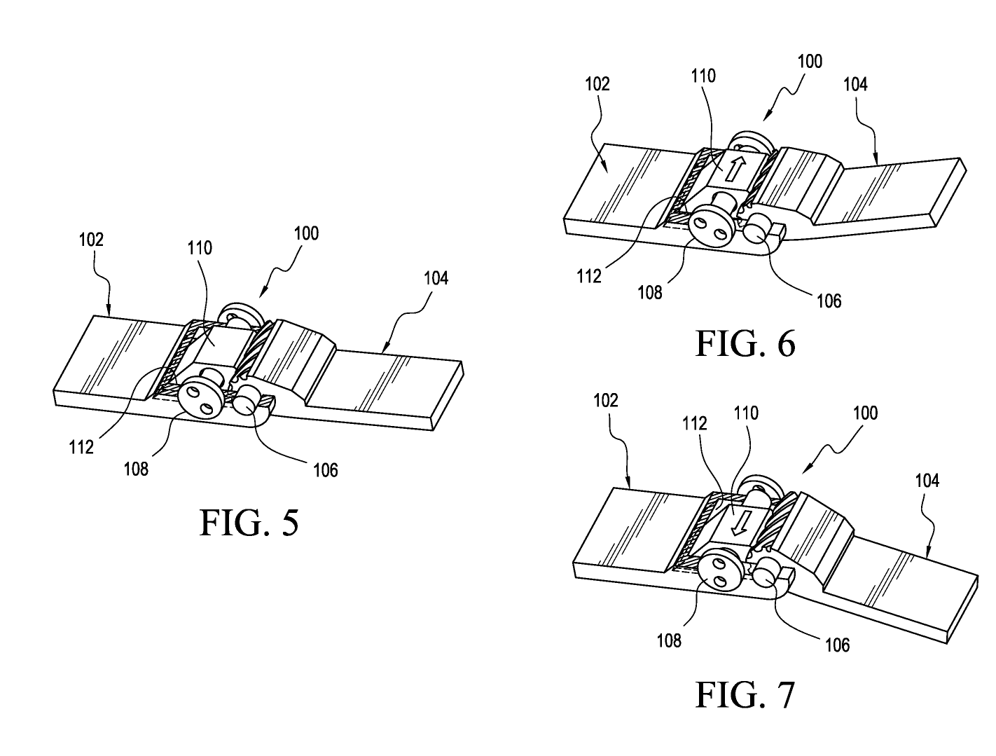

Referring now to FIGS. 4-9, an embodiment of a hinge assembly 100 includes first and second connection members 102, 104, a shaft member 108, and a translating member 110 (shown in FIG. 5). At least one of the first and second hinge connection members 102, 104 is arranged to rotate relative to the other about at least one pivot point 106.

As seen in FIGS. 4 and 4A, the shaft member 108 is connected to the first connection member 102. The shaft member 108 can be rotatably connected to the first connection member 102. The shaft member 108 can be located on a different axis than the pivot point 106. The shaft member 108 can be located on a same axis as the pivot point 106. The shaft member 108 can be a set screw or another suitable member.

The translating member 110 is operatively connected to the shaft member 108. In an embodiment, the translating member 110 can be attached to the shaft member 108 and positioned within a receiving space 112 defined by the first connection member 102. The translating member 110 can be attached to the shaft member 108 via a threaded connection 111. The translating member 110 is restrained from rotation so that rotation of the shaft member 108 results in translation of the translating member 110 along the shaft member 108. The translating member 110 can be restrained from rotation by the first connection member 102. The translating member 110 is also operatively connected to an end portion of the second connection member 104 so that translation of the translating member results in rotation of the second connection member 104 about the pivot point 106.

The operation of the hinge assembly 100 according to an embodiment will now be described. FIG. 5 shows the hinge assembly 100 in a neutral position. When the shaft member 108 is rotated in a counterclockwise direction, the shaft member 108 drives the translating member 110 to translate in a first direction along a length of the shaft member 108 as shown in FIG. 6. The translation of the translating member 110 in the first direction in turn drives the second connection member 104 to rotate in the counterclockwise direction about the pivot point 106 from the zero position. Optionally, the shaft member 108 can be configured to receive a tool member such that a clinician or wearer can use a tool member to manually rotate the shaft member 108 as desired.

When the shaft member 108 is rotated in a clockwise direction, the shaft member 108 drives the translating member 110 to translate in a second direction opposite the first direction along a length of the shaft member 108 as shown in FIG. 7. The translation of the translating member 110 in the second direction in turn drives or causes the second connection member 104 to rotate in the clockwise direction about the pivot point 106 from the zero position. It will be appreciated that other movements of the hinge assembly 100 are possible. For instance, clockwise rotation of the shaft member 108 can result in counter-clockwise rotation of the second connection member 104.

Rotation of the shaft member 108 thus both controls and drives the angular movement of the second connection member 104 relative to the first connection member 102. Because of high shear friction and/or the high mechanical advantage of the hinge assembly 100, the hinge assembly 100 is self-locking such that it can only be unlocked and adjusted by turning the shaft member 108. As such, whatever angle is set by the shaft member 108 remains substantially fixed until the shaft member 108 is readjusted via an input force or torque applied to the shaft member 108. This has the effect of allowing the hinge 100 to be locked and/or unlocked under a load, increasing the ease of use. The hinge 100 can also be adjusted in different rotational directions from the zero position.

Furthermore, because rotation of the shaft member 108 and corresponding translation of the translating member 110 drives and controls rotation of the second connection member 104, the hinge assembly 100 is continuously adjustable within a range of motion defined by the hinge assembly 100. This continuous adjustability in combination with the self-locking configuration of the hinge assembly 100 also allows the hinge assembly 100 to be locked in an infinite number of positions within the range of motion rather than only being lockable in discrete increments as in the prior art, providing greater control of movement and functionality.

It will be appreciated that the range of motion of the hinge assembly 100 can be defined at least in part by the pitch and/or lead of the shaft member 108. The range of motion can also be defined at least in part by the tooth angle or orientation of one or more teeth (described below) on the translating member 110 or the second connection member 104. In other embodiments, the range of motion of the hinge assembly 100 can also be defined at least in part on the configuration and dimensions of the shaft member 108, the translating member 110, the receiving space 112, and/or the end portion of the second connection member 104.

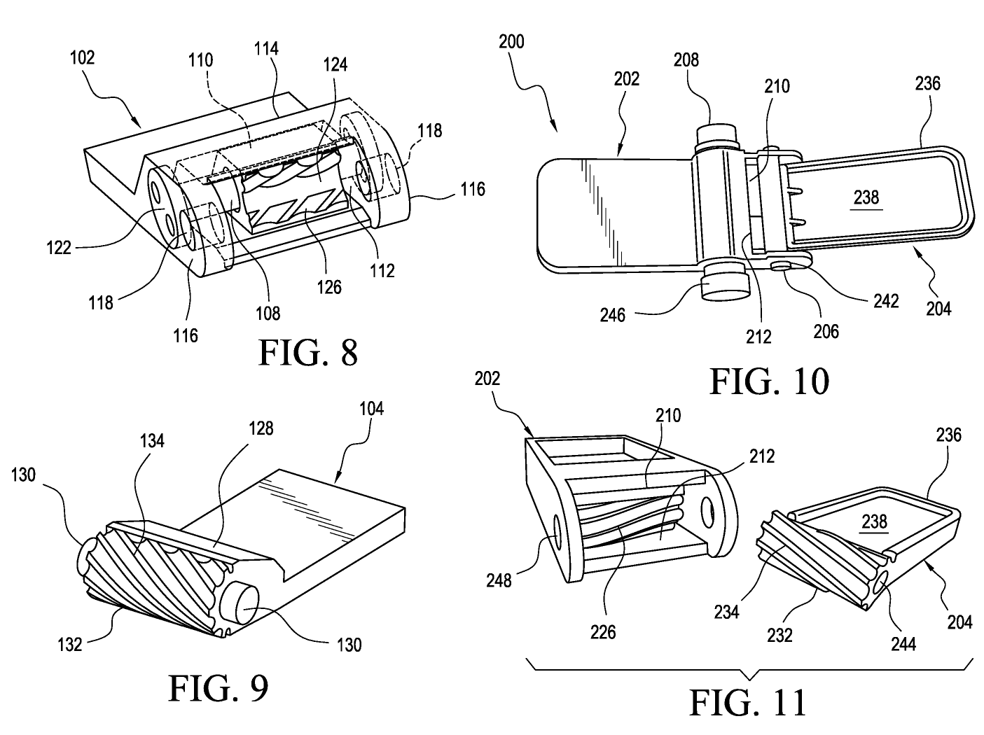

Referring now to FIG. 8, the first connection member 102 includes an end portion 114 defining support arm portions 116 on opposite sides of the receiving portion 112. A pair of opposing holes 118 are formed in the support arm portions 116. The shaft member 108 is rotatably connected to the first connection member 102 via a through-hole intersecting the receiving space 112. According to a variation, the through-hole can be countersunk so that opposing head portions 122 of the shaft member 108 are generally flush with the outer surface of the support arm portions 116. The end portion 114 can have an increased thickness for accommodating the translating member 110 and the shaft member 108.

The translating member 110 can be threadedly attached to the shaft member 108 in the receiving space 112. The translating member 110 is adapted to move axially on threads of the shaft member 108 when the shaft member 108 rotates. When the translating member 110 is attached to the shaft member 108 it is captured within the receiving space 112. More particularly, the translating member 110 is captured between the support arm portions 116 and a top and bottom wall defining the receiving space 112. This restricts or prevents relative rotation between the translating member 110 and the first connection member 102. It also provides a solid connection between the translating member 110 and the first connection member 102. The translating member 110 is dimensioned and configured such that it can move back and forth between sidewalls of the receiving space 112.

The translating member 110 is shown as a block member but can be any suitable member such as a nut member or a threaded sleeve. In an embodiment, the translating member 110 can include generally flat top and bottom surfaces, and an angled back wall extending downwardly and backward from the top surface toward the bottom surface. This shape can cooperate with the shape of the receiving space 112 to help restrain the translating member 110 from rotation within the receiving space 112.

A face 124 of the translating member 110 is arranged to mesh or interact with the second connection member 104. In the illustrated embodiment, the face 124 defines a concave curvature and a plurality of teeth 126. The teeth 126 can be formed at an angle to the face 124 and can extend or curve across a width of the face 124. The teeth 126 can extend completely or along one or more portions of the face 124. The teeth 126 can be generally helical. It will be appreciated that the translating member 110 can have any suitable number of teeth. For instance, the face 124 can include three, four, five, or any other number of teeth.

According to a variation, the translating member 110 can be selectively removable from the hinge assembly 100. This can allow the translating member 110 to be replaced if damaged without having to replace the entire hinge assembly 100. This can also allow the hinge assembly 100 to be customized by exchanging or swapping out the translating member 110 for different translating members. For instance, by changing the translating member, the hinge assembly 100 can be customized for a desired treatment protocol, patient characteristics, and/or other factors.

Referring to FIGS. 8 and 9, the second connection member 104 includes an end portion 128 including a pair of opposing pin members 130. The pivot point 106 (shown in FIG. 7) can be defined by the pin members 130 of the second connection member 104 inserted in the openings 118 of the first connection member 102 but other pivot points are possible. The end portion 128 of the second connection member 104 is sized and configured to be positioned between the support arm portions 116 of the first connection member 102.

A face 132 of the second connection member 104 is arranged to interact or mesh with the face of the translating member 110. The end portion 128 or the face 132 defines a convex or cylindrical curvature arranged to fit the curvature of the translating member 110. A plurality of teeth 134 are arranged to mesh with the teeth 126 of the translating member 110. The teeth 134 can be formed at an angle to the face 132 and can extend or curve across a width of the face 132. The teeth 134 can be generally helical. The teeth 134 can be oriented in an opposite direction from the teeth 126. The face 132 can include between about 3 and about 12, about 4 and about 10 (e.g. about 5), or about 5 and about 8 teeth. In other embodiments, the face 132 can include more or less teeth.

The interaction or tooth loads between the translating member 110 and the second connection member 104 create a driving force on the second connection member 104 as the translating member 110 translates along the shaft member 108. As the translating member 110 moves along the length of the shaft member 108, the interaction between the teeth 126, 134 generates the driving force that in turn rotates the second connection member 104 about the pivot point 106. The end portion 128 can have an increased diameter or thickness to better accommodate the teeth 134 and/or the interaction between the first and second connection members 102, 104.

It will be appreciated that the dimension and configuration of the interaction between the translating member 110 and the second connection member 104 can at least in part define the strength of the hinge assembly 100. For instance, the length, angle, depth, thickness, curvature, pressure angle, and/or pitch of the teeth can at least in part define the strength of the hinge assembly 100.

In an embodiment, the teeth 126 of the translating member 110 are engaged with the teeth 134 of the second connection member 104 along substantially the entire length of the teeth 126 extending in a direction across the translating member 110. In addition, the face 124 of the translating member 110 at least partially wraps around the face 132 of the second connection member 104, increasing the contact area between the translating member and the second connection member 104. This greater contact area helps form a solid connection between the translating member 110 and the second connection member 104, which, in turn, helps the hinge assembly 100 to distribute greater loads.

As such, the hinge 100 can be made smaller and/or simpler than in the prior art. For instance, the first connection member 102, the second connection member 104, and/or the translating member 110 can be made from a plastic material or other lightweight material that can resist deformation during use. This can result in hinge assemblies that are more cost effective to manufacture, less bulky, lighter-weight, and more comfortable to wear. It will be appreciated that the shaft member 108 may be formed of metal, plastic, or any other suitable material. Furthermore, the first connection member 102, the second connection member 104, and/or the translating member 110 can be formed of metal or carbon fiber.

The interaction between the translating member 110 and the second connection member 104 also substantially reduces gaps between the translating member 110 and the second connection member 104. This has the effect of reducing lost motion or play in the hinge assembly 100, which, in turn, improves the ability of the hinge assembly 100 to control movement.

FIGS. 10 and 11 illustrate another embodiment of a hinge assembly 200. The hinge assembly 200 can be similar to the hinge assembly 100. For instance, the hinge assembly 200 can include first and second connection members 202, 204 which are arranged to rotate relative to another about at least one pivot point 206.

The first and/or second connection members 202, 204 can be formed of plastic or another lightweight, high strength material. Optionally, at least one of the first or second connection members 202, 204 can include a peripheral rim 236 surrounding a recessed center portion 238. This has the effect of giving the connection member a greater overall thickness while using less material, reducing the overall weight of the connection member.

The pivot point 206 can be defined by a pin member 242 extending through pin holes 248 in the first connection member 202 and a pin hole 244 defined through the second connection member 204. The end portion of the second connection member 204 can fit between arm portions defined by the first connection member 202.

A shaft member 208 is rotatably connected to the first connection 202. The shaft member 208 can be located on a different axis than the pivot point 206. The shaft member 208 includes a threaded portion and opposing socket heads 246, each arranged to receive a tool member (e.g., a hex key) such that a clinician or wearer can more easily turn the shaft member 208. Optionally, the socket heads 246 can include a textured or knurled outer surface, providing an improved gripping surface. The shaft member 208 can be formed of metal or any other suitable material.

A translating member 210 comprising a block member is threadedly attached to the shaft member 208 and positioned within a receiving space 212 defined by the first connection member 202. The translating member 210 is restrained from rotation within the receiving space 212 so that rotation of the shaft member 208 results in translation of the translating member 210 along the shaft member 208. The translating member 210 can be formed of plastic or any other suitable material.

The translating member 210 engages or meshes with an end portion of the second connection member 204 so that translation of the translating member results in rotation of the second connection member 204 about the pivot point 206. The translating member 210 can define a concave curvature and a plurality of teeth 226. The teeth 226 can be formed at an angle to the face of the translating member 210 and can extend in a direction across a width of the face.

An end portion of the second connection member 204 is arranged to interact with the teeth of the translating member 210. The end portion defines a convex or cylindrical curvature arranged to fit the curvature of the translating member 210. A plurality of teeth 234 are arranged to mesh with the teeth 226 of the translating member 210. The teeth 234 can be formed at an angle to the face of the second connection member 204 and can extend or curve in a direction across a width of the face 232. The teeth 234 can have a different orientation from the teeth 226. Tooth loads between the translating member 210 and the second connection member 204 can generate a driving force when the translating member 210 translates to rotate the second connection member 204. The end portions of the first and second connection members 202, 204 can have an increased diameter or thickness to help accommodate the interaction between the connection members and the translating member 210.

To adjust the angular position between the first and second connection members 202, 204, the shaft member 208 is rotated relative to the first connection member 202. This rotation causes or drives the translating member 210 to translate along the shaft member 208, which, in turn, drives the second member to rotate relative to the first connection member 204 about the pivot point 206. The hinge 200 is self-locking such that whatever angle is set by the shaft member 208 remains substantially fixed until the shaft member 208 is readjusted.

It will be appreciated that the hinge assemblies described are to be regarded as exemplary only, as other hinge assemblies are possible. For instance, the hinge assembly can include one, two, three, or any other suitable number of pivot points. In other embodiments, the shaft member can be attached to a central member and the translating member can define teeth on opposing sides of the translating member. The first and second connection members can be pivotally attached to opposing sides of the central member. The first and second connection members can each define a plurality of teeth arranged to mesh with the teeth of the central member. As such, when the translating member is driven along a length of the shaft member, the engagement between the translating member and the first and second connection members drives rotation of both the first and second connection members. In other embodiments, the shaft member and/or translating member may have larger diameters. In other embodiments, the second connection member can define the receiving space and/or the shaft member can be rotatably connected to the second connection member.

In other embodiments, the translating member and/or second connection member can have more or less teeth or different helix angles. Moreover, while helical teeth are described, it will be appreciated that other gearing and/or arrangements are possible to rotate at least one of the connection members upon axial movement of the translating member. In yet other embodiments, translation of the translating member along the shaft member drives rotation of the first connection member relative to the second connection member. While the connection members are described as being separate from the orthopedic or prosthetic device, in other embodiments, the connection members can be integral to the orthopedic or prosthetic device. For instance, the connection members may be integral to a strut assembly of an orthopedic device. It will also be appreciated that the connection members can have any suitable shape and/or size.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

1. A hinge assembly for use in an orthopedic or prosthetic system, the hinge assembly comprising:
first and second connection members arranged to rotate relative to one another about at least one pivot point;
at least one shaft member rotatably attached to the first connection member; and
a translating member attached to the at least one shaft member and operatively connected to or meshing with the second connection member,
wherein rotation of the at least one shaft member drives translation of the translating member along a length of the shaft member and translation of the translating member along the shaft member drives rotation of the second connection member relative to the first connection member.

2. The hinge assembly of claim 1, wherein the hinge assembly is self-locking such that an angle defined between the first and second connection members is only adjustable via an input force applied to the at least one shaft member.

3. The hinge assembly of claim 1, wherein the translating member defines a first plurality of teeth and the second connection member defines a second plurality of teeth arranged to mesh with the first teeth.

4. The hinge assembly of claim 3, wherein the first and second teeth comprise helical teeth.

5. The hinge assembly of claim 4, wherein the first teeth are oriented in a different direction from the second teeth.

6. The hinge assembly of claim 4, wherein tooth loads between the first and second teeth drive rotation of the second connection member relative to the first connection member when the translating member translates along the at least one shaft member.

7. The hinge assembly of claim 3, wherein the translating member includes a concave face defining the first teeth.

8. The hinge assembly of claim 7, wherein the second connection member includes an end portion having a convex face defining the second teeth and arranged to fit the concave face of the translating member.

9. The hinge assembly of claim 1, wherein the translating member comprises a block member.

10. The hinge assembly of claim 9, wherein the block member includes a back wall extending at an angle between generally flat upper and lower surfaces.

11. The hinge assembly of claim 1, wherein the shaft member comprises a set screw.

12. The hinge assembly of claim 1, wherein the first and second connection members are formed of a plastic material.

13. The hinge assembly of claim 1, wherein the first and second connection members are pivotally connected via pin members.

14. The hinge assembly of claim 1, wherein the translating member is threadedly attached to the at least one shaft member.

15. The hinge assembly of claim 1, wherein the translating member is disposed in a receiving space defined by the first connection member.

16. An orthopedic system comprising:
first and second struts; and
a hinge assembly connecting the first and second struts, the hinge assembly comprising:
a first connection member connected to the first strut; and
a second connection member connected to the second strut and arranged to rotate relative to first connection member about at least one pivot point;
at least one shaft member rotatably attached to the first connection member; and
a translating member attached to the at least one shaft member and operatively connected to or meshing with the second connection member,
wherein rotation of the at least one shaft member drives translation of the translating member along a length of the shaft member and translation of the translating member along the shaft member drives rotation of the second connection member relative to the first connection member.

17. The orthopedic system of claim 16, wherein the hinge assembly is self-locking such that an angle defined between the first and second struts is only adjustable via an input force applied to the at least one shaft member.

18. The orthopedic system of claim 16, wherein the translating member comprises a block member disposed in a receiving space defined by the first connection member.

19. The orthopedic system of claim 16, wherein the translating member defines a first plurality of teeth and the second connection member defines a second plurality of teeth arranged to mesh with the first teeth.

20. A hinge assembly for use in an orthopedic or prosthetic system, the hinge assembly comprising:
first and second connection members arranged to rotate relative to one another about at least one pivot point;
at least one shaft member rotatably attached to the first connection member; and
a translating member attached to the at least one shaft member and operatively connected to or meshing with the second connection member, the translating member comprising a block member disposed in a receiving space defined by the first connection member,
wherein rotation of the at least one shaft member drives translation of the translating member along a length of the shaft member within the receiving space and translation of the translating member along the shaft member drives rotation of the second connection member relative to the first connection member.
